(19) United States
(12) Patent Application Publication
(10) Pub. No.: US 2024/0335218 A1
(43) Pub. Date: Oct. 10, 2024

(54) ADJUSTABLE-LENGTH BONE FIXATION DEVICE AND ASSEMBLING METHOD THEREOF

(71) Applicants: Taipei Medical University, Taipei (TW); National Taipei University of Technology, Taipei (TW)

(72) Inventors: YI-JIE KUO, Taipei (TW); Yu-Pin Chen, Taipei (TW); Shu-Wei Huang, Taipei (TW); Chung-Lei Hsiung, Taipei (TW); Cho-Pei Jiang, Taipei (TW)

(21) Appl. No.: 18/626,341

(22) Filed: Apr. 4, 2024

(30) Foreign Application Priority Data
Apr. 7, 2023 (TW) 112113155

(51) Int. Cl. A61B 17/86; A61B 17/68; A61B 17/74

(52) U.S. Cl. CPC A61B 17/864; A61B 17/742; A61B 17/866; A61B 17/8685; A61B 2017/681

(57) ABSTRACT

A device is provided. The device includes an external screw includes a first channel extending along a longitudinal axis, and the first channel including a first inner side including at least one protrusion; and an internal screw includes a first pushing rod section including a first threaded section and at least one groove, the at least one groove is configured to accommodate the at least one protrusion of the first inner side, and the first pushing rod section is configured to slide within the first channel along the longitudinal axis.

### CROSS-REFERENCE TO RELATED APPLICATION

The present application claims the benefit of and priority to Taiwan Patent Application Ser. No. 112113155, filed on Apr. 7, 2023, entitled “A SLIDING DEVICE FOR BONE FRACTURE”, the contents of which are hereby incorporated herein fully by reference into the present application for all purposes.

### FIELD

The present disclosure relates to a device for bone fracture, and more particularly, to a device that changed the overall length by sliding.

### BACKGROUND

Research has found that elderly patients with osteoporosis and other stress factors by the patient may experience a certain degree of femoral neck shortening during the healing process of femoral neck fractures due to impact at the fracture site. The average femoral neck length can be shortened by 0.43 cm (range: 0 to 2.15 cm). In accordance with this phenomenon, it has been found that if multiple compression bone screws are inserted parallel to each other, the screws would retreat, with an average displacement of 0.33 cm (range: 0 to 2.43 cm). Conversely, if multiple compression bone screws are inserted in a divergent manner, it will be more likely to cause serious complications such as screw penetration of the femoral head because the screws cannot adapt to the femoral neck healing and shortening process.

For patients with severe osteoporosis who have a femoral neck fracture, surgery is usually performed with divergent compression bone screws. However, postoperative follow-up has shown that due to the shortening of femoral neck due to the inability to cooperate with the impaction of the healing process of the femoral neck, one of the bone screws may penetrate the femoral head. Such complications lead to severe hip cartilage wear and pain during walking. In addition, osteoporosis patients who undergo femoral neck fracture fixation with three parallel compression bone screws may experience obvious bone screws migration as the bone screws follow the femoral neck shortening during the healing process. As a result, patients often suffer from muscle and ligament irritation due to the displaced bone screws and are often unable to sleep on their sides or suffer from pain on the external side of the hip when moving. Some patients may even need a second surgery to remove the migrated bone screws due to muscle and ligament compression symptoms caused by bone screw migration.

In view of the above discussion, the current problems and risks caused by using compression screws for femoral neck fracture fixation are the shortening of the femoral neck that may occur during the healing process of femoral neck fractures, which in turn may cause the displacement of the bone screws during the healing of the femoral neck fracture. Therefore, improving the treatment of femoral neck fractures and reducing the complications caused by the placement of bone screw are urgently needed in the art of treating patients with osteoporosis.

### SUMMARY

In a first aspect of the present disclosure, a device is provided. The device includes an external screw includes a first channel extending along a longitudinal axis, and the first channel including a first inner side including at least one protrusion; and an internal screw includes a first pushing rod section including a first threaded section and at least one groove, the at least one groove is configured to accommodate the at least one protrusion of the first inner side, and the first pushing rod section is configured to slide within the first channel along the longitudinal axis.

In an implementation of the first aspect of the present disclosure, the first pushing rod section of the internal screw further includes a second channel, the second channel includes a second inner side, and the first threaded section is located on the second inner side.

In an implementation of the first aspect of the present disclosure, the second channel is configured to accommodate a length adjuster, the length adjuster includes a knob, a smooth section and a second threaded section, and the second threaded section is compatible with the first threaded section.

In an implementation of the first aspect of the present disclosure, the first channel includes a first region and a second region, a diameter of the first region is greater than a diameter of the second region, and the diameter of the second region is smaller than a diameter of the knob.

In an implementation of the first aspect of the present disclosure, the external screw includes a first continuous protrusion part, and the internal screw includes a second continuous protrusion part.

In an implementation of the first aspect of the present disclosure, the first continuous protrusion part and the second continuous protrusion part include screw threads.

In an implementation of the first aspect of the present disclosure, a first spacing of the first continuous protrusion part is different from a second spacing of the second continuous protrusion part.

In an implementation of the first aspect of the present disclosure, a spacing ratio of the first continuous protrusion part to the second continuous protrusion part is 1:2.

In an implementation of the first aspect of the present disclosure, the first inner side in the first channel forms a circumference, and the circumference includes a plurality of protrusions distributed evenly.

In an implementation of the first aspect of the present disclosure, the plurality of protrusions is 4 protrusions.

In an implementation of the first aspect of the present disclosure, the first pushing rod section of the internal screw forms a circumference, and the circumference includes a plurality of grooves distributed evenly.

In an implementation of the first aspect of the present disclosure, the plurality of grooves is 4 grooves.

In an implementation of the first aspect of the present disclosure, an adjustable sliding distance of the internal screw relative to the external screw is no more than 2 cm.

In an implementation of the first aspect of the present disclosure, the first thread section of the first pushing rod section includes external screw threads.

In an implementation of the first aspect of the present disclosure, a material of the internal screw and the external screw includes Ti-6Al-4V.

In an implementation of the first aspect of the present disclosure, a material of the internal screw and the external screw includes stainless steel.

In a second aspect of the present disclosure, an assembling method is provided. The device including an internal screw and an external screw, the external screw including a first channel configured to accommodate a length adjuster, the first channel, a first inner side of the first channel includes at least one protrusion, the internal screw including a first pushing rod section with at least one groove, the first pushing rod section including a second channel, a second inner side of the second channel including internal screw threads, the at least one groove configured to accommodate the at least one protrusion, and the method including: inserting the first pushing rod section of the internal screw into the first channel of the external screw, the internal screw and the external screw being slidable relative to each other; placing the length adjuster in the first channel of the external screw, the length adjuster being provided with a second thread section which includes external screw threads, and the external screw threads on the second thread section being made compatible with the internal screw threads of the first pushing rod section; rotating the length adjuster to adjust a sliding distance of the internal screw relative to the external screw; and removing the length adjuster.

### BRIEF DESCRIPTION OF THE DRAWINGS

Aspects of the present disclosure are best understood from the following detailed description when read with the accompanying figures. Various features are not drawn to scale. Dimensions of various features may be arbitrarily increased or reduced for clarity of discussion.

FIG. 1 is a stereoscopic view illustrating a sliding device for treating bone fracture, according to an example implementation of the present disclosure.

FIG. 2A is a sectional view illustrating an external screw according to an example implementation of the present disclosure.

FIG. 2B is a stereoscopic view illustrating an internal screw according to an example implementation of the present disclosure.

FIG. 3 is a first schematic diagram illustrating an assembly of the sliding device for treating bone fracture according to an example implementation of the present disclosure.

FIG. 4 is a second schematic diagram illustrating an assembly of the sliding device for treating bone fracture according to an example implementation of the present disclosure.

FIG. 5 is a cross-sectional view illustrating the assembly of the sliding device in FIG. 4 for treating bone fracture according to an example implementation of the present disclosure; and

FIG. 6 is a flowchart illustrating the assembly of a sliding device for bone fracture according to an example implementation of the present disclosure.

### DETAILED DESCRIPTION OF THE PREFERRED EMBODIMENTS

The following description will refer to the accompanying drawings to describe the present disclosure more fully. Illustrated in the drawings are exemplary embodiments of the present disclosure. This disclosure may, however, be embodied in many different forms and should not be construed as limited to the exemplary embodiments set forth herein. Rather, these exemplary embodiments are provided so that this disclosure will be thorough and complete and will fully convey the scope of the disclosure to those skilled in the art. Similar reference numerals designate the same or similar elements.

The terminology used herein is for the purpose of describing exemplary embodiments only and is not intended to limit the present disclosure. As used herein, the singular forms “a”, “an” and “the” are intended to include the plural forms as well, unless the context clearly dictates otherwise. Furthermore, when used herein, “comprises” and/or “comprises” or “comprises” and/or “comprises” or “has” and/or “has”, integers, steps, operations, elements and/or components, but does not preclude the presence or addition of one or more other features, regions, integers, steps, operations, elements, components and/or groups thereof.

Unless otherwise defined, all terms (including technical and scientific terms) used herein have the same meaning as commonly understood by one of ordinary skill in the art to which this disclosure belongs. Furthermore, unless clearly defined herein, terms such as those defined in commonly used dictionaries should be construed as having meanings consistent with their meanings in the related art and this disclosure and will not be construed as idealistic or overly formal meaning.

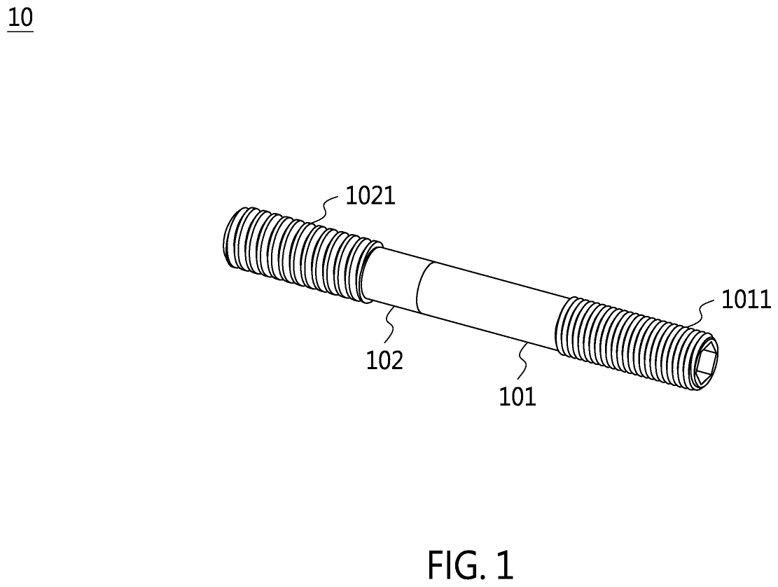

The exemplary embodiments will be described with reference to the drawings in FIGS. 1 to 6. Detailed description will be described in detail with reference to the accompanying drawings of the present invention. The components depicted therein need not be drawn to scale. Furthermore, through multiple views, similar or identical reference numerals in the drawings represent similar or identical components.

FIG. 1 is a stereoscopic view illustrating a sliding device for bone fracture, according to an example implementation of the present disclosure.

With reference to FIG. 1, the sliding device for bone fracture 10 may include an external screw 101 and an internal screw 102.

In some implementations, the internal screw 102 and the external screw 101 may be made of Ti-6Al-4V. In some embodiments, the internal screw 102 and the external screw 101 are made of stainless steel. However, the present invention is not limited thereto, and those skilled in the art can adjust the materials of the internal screw 102 and the external screw 101 according to actual needs.

In some implementations, to enhance the bone integration of the sliding device for bone fracture, nano-sized hydroxyapatite powder is laser-deposited on the portion of the sliding device for bone fracture 10, which is for bone integration to accelerate the attachment of bone cells to the surface. Therefore, the surfaces of the external and internal screws are covered with nano-sized hydroxyapatite powder.

Please return to FIG. 1, the external screw 101 is able to accommodate part of the internal screw 102. The external screw 101 has a first continuous protrusion part 1011 and the internal screw 102 has a second continuous protrusion part 1021. Since the sliding device for bone fracture 10 has the first continuous protrusion part 1011 and the second continuous protrusion part 1021, the sliding device for bone fracture and the bone can be closely contacted to help the bone occlusion and increase the probability of bone fracture healing.

In some implementations, the first continuous protrusion part 1011 and the second continuous protrusion part 1021 include screw threads.

In some implementations, the first protrusion part 1011 may have a first spacing and the second continuous protrusion part 1021 may have a second spacing. The first spacing of the first continuous protrusion part 1011 is different from the second spacing of the second continuous protrusion part 1021. For example, the spacing of the second continuous protrusion part 1021 is sparser than that of the first continuous protrusion part 1011. The design of the second continuous protrusion part 1021 with a sparser spacing compared to the first continuous protrusion part 1011 avoid excessive bone destruction and excessive pressure. Therefore, it is possible to reduce the excessive pressure caused by the placement of the sliding device for bone fracture 10 in the bone, resulting in collapse and deformation of the bone.

In some implementations, the spacing ratio between the first continuous protrusion part 1011 and the second continuous protrusion part 1021 is ratio 1:2.

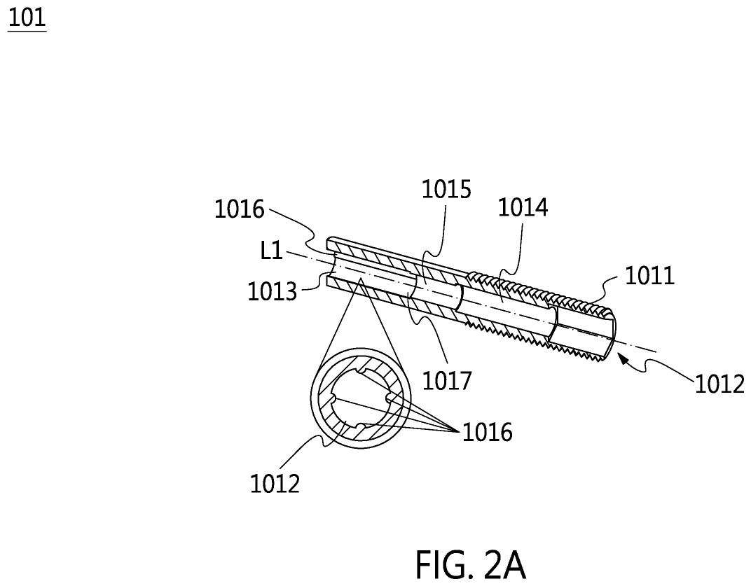

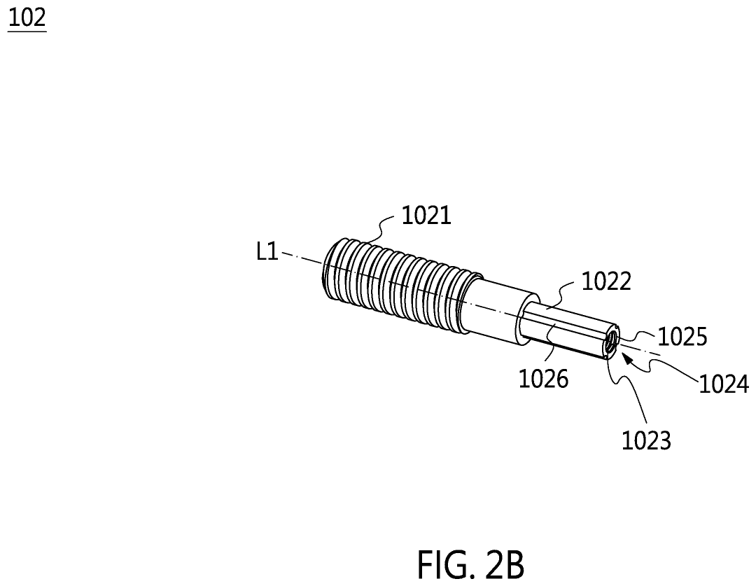

FIG. 2A is a sectional view illustrating an external screw according to an example implementation of the present disclosure. FIG. 2B is a stereoscopic view illustrating an internal screw according to an example implementation of the present disclosure.

With reference to FIG. 2A, the external screw 101 includes a first continuous protrusion part 1011 and a first channel 1012 extending along the longitudinal axis L1. The first channel 1012 includes a first inner side 1013 and further comprises a first region 1014, a second region 1015, and a third region 1017. The diameter of the first region 1014 is greater than the diameter of the second region 1015.

In some implementations, the first inner side 1013 comprises at least one protrusion 1016.

In some implementations, the first inner side 1013 forms a circumference, which comprises a plurality (e.g., 4) protrusions 1016 equally spaced in the circumferential direction.

With reference to FIG. 2B, the internal screw 102 comprises a second continuous protrusion part 1021 and a first pushing rod section 1022, wherein the first pushing rod section 1022 comprises a first thread section 1023.

In some implementations, the first thread section 1023 includes internal screw threads. Specifically, the first pushing rod section 1022 comprises a second channel 1024 extending along the longitudinal axis L1, the second channel 1024 further includes a second inner side 1025, and the second inner side 1025 includes the internal screw thread. In another embodiments, the first thread section 1023 of the first pushing rod section 1022 includes external screw threads.

In some implementations, the first pushing rod section 1022 of the internal screw 102 includes at least one groove 1026. The groove 1026 is designed to accommodate the protrusion 1016 of the first inner side 1013, ensuring the internal screw 102 and the external screw 101 do not rotate relative to each other. Additionally, the first pushing rod section 1022 is slidable within the first channel 1012 along the longitudinal axis.

In some implementations, the first pushing rod section 1022 of the internal screw 102 forms a circumference, which further comprises a plurality of (e.g., 4) grooves 1026 equally spaced in the circumferential direction.

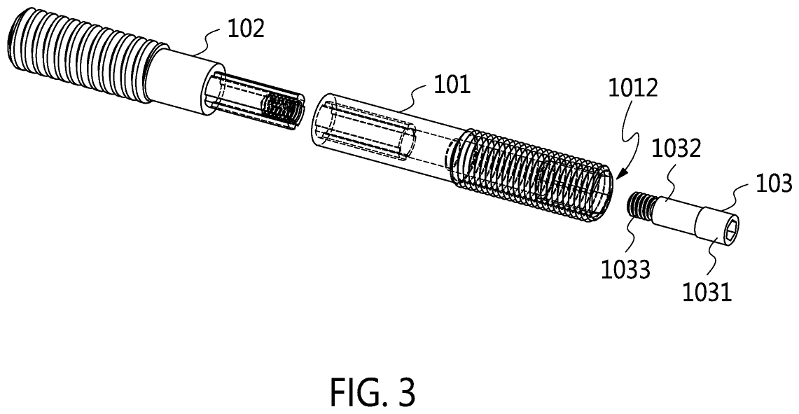

FIG. 3 is a first schematic diagram illustrating the assembly of a sliding device for bone fracture according to an example implementation of the present disclosure.

With reference to FIG. 3, the sliding device for bone fracture 10 comprises an internal screw 102 and an external screw 101. In addition, a length adjuster 103 is prepared to adjust the relative length between the internal screw 102 and an external screw 101 more precisely. The length adjuster 103 comprises a knob 1031, a smooth section 1032 and a second thread section 1033.

In some implementations, the length adjuster 103 is made of Ti-6Al-4V. In some embodiments, the length adjuster 103 is made of stainless steel. However, the present invention is not limited thereto, and those skilled in the art can adjust the materials of the length adjuster 103 according to actual needs.

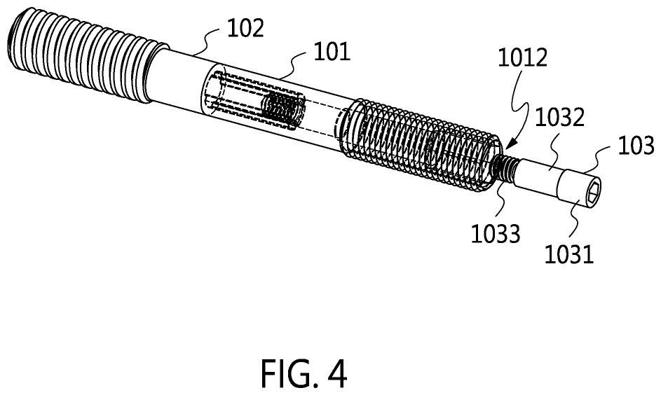

FIG. 4 is a second schematic diagram illustrating the assembly of a sliding device for bone fracture according to an example implementation of the present disclosure.

With reference to FIG. 4, the external screw 101 includes a first channel 1012 accommodating the length adjuster 103, and the first channel 1012 further includes a first inner side 1013 having at least one protrusion 1016. The internal screw 102 includes a first pushing rod section 1022 having at least one groove 1026, and the first pushing rod section 1022 may be inserted into the first channel 1012 of the external screw 101. The internal screw 102 and the external screw 101 are slidable relative to each other.

In some implementations, the first pushing rod section 1022 further includes a second channel 1024, the second channel 1024 includes a second inner side 1025, and the second inner side 1025 has a first thread section 1023. The second thread section 1033 of the length adjuster 103 is compatible with the internal screw threads of the first thread section 1023. Therefore, after the length adjuster 103 extends into the first channel 1012, the second thread section 1033 is compatible with the first thread section 1023 of the first pushing rod section 1022. However, in other implementations where the first thread section 1023 includes external screw threads, the second thread section 1033 of the length adjuster 103 is compatible with the external screw threads of the first thread section 1023.

It should be noted that compatibility between internal and external screw threads means that these two threads may be coupled to each other. For example, when the screw thread diameter and pitch of the internal and external screw threads are the same, it indicates that these two threads are compatible. In some implementations, the external screw threads may be referred to male screw threads and the internal screw threads may be referred to female screw threads.

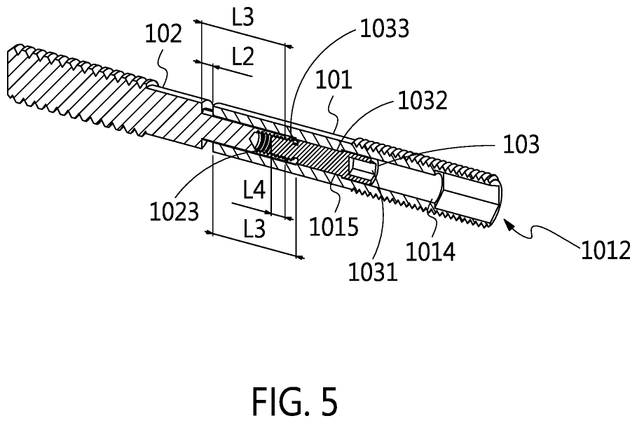

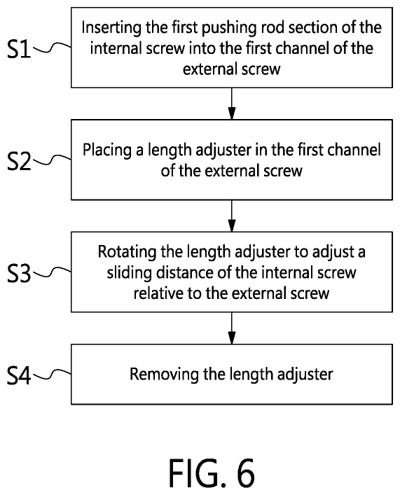

FIG. 5 is a cross-sectional view illustrating the assembly of a sliding device for bone fracture according to an example implementation of the present disclosure. FIG. 6 is a flowchart illustrating the assembly of a sliding device for bone fracture according to an example implementation of the present disclosure.

With reference to FIG. 5 and FIG. 6. In step S1, inserting the first pushing rod section 1022 of the internal screw 102 into the first channel 1012 of the external screw 101. In step S2, putting the length adjuster 103 into the first channel 1012 of the external screw 101. More specifically, the second region 1015 of the external screw 101 accommodates the smooth section 1032 of the length adjuster 103, and the first region 1014 accommodates the knob 1031 of the length adjuster 103. In addition, since the diameter of the knob 1031 of the length adjuster 103 is larger than that of the second region 1015, the knob of the length adjuster 103 may not enter the second region 1015. In step S3, rotating the length adjuster 103 to adjust the relative distance of the internal screw 102 and the external screw 101. When the length adjuster 103 is turned clockwise or counterclockwise by the knob 1031, the internal screw 102 may be moved forward or backward toward the first channel 1012 of the external screw 101. In some embodiments, the length adjuster 103 assists to adjust the relative sliding distance L2 between the internal screw 102 and the external screw 101. The first pushing rod section 1022 of the internal screw 102 and the third region 1017 of the external screw 101 have the same length L3 (e.g., 2.5 cm). When the second thread section 1033 of the length adjuster 103 is 1.5 cm, and the desired relative sliding distance to adjust between the internal screw and the external screw is 1.0 cm. Then the length L4 of the external thread of the length adjuster needs to screw into the internal screw with internal thread is 0.5 cm.

In some implementations, the designed internal screw 102, external screw 101, and length adjuster 103 ensure that the relative sliding distance L2 between the internal screw 102 and the external screw 101 does not exceed 2.0 cm. For example, the first pushing rod section 1022 of the internal screw 102 has the same length as the third region 1017 of the external screw 101 (e.g., 2.5 cm), while the second thread section 1033 of the length adjuster 103 is 2.1 cm. To prevent at least one groove 1026 of the first pushing rod section 1022 of the internal screw 102 and at least one protrusion 1016 of the first inner side 1013 of the external screw 101 from being accommodated with insufficient overlap length, resulting in easy separation of the internal and external screws after removal of the length adjuster. The second thread section 1033 of the length adjuster 103 needs to be screwed in at least 0.1 cm to achieve the sliding device for bone fracture 10 in which the sliding distance of the internal screw 102 compared with the external screw 101 does not exceed 2.0 cm. It should be noted that the detachment of the internal and external screws refers to the fact that the two screws do not contact with each other. For example, the groove of the internal screw and the protrusion of the external screw do not touch each other, indicating that the internal and external screws are detached. However, the present invention is not limited to this, and those skilled in the art can adjust the length of the internal screw 102, the external screw 101, and the length adjuster 103 according to actual needs as usual knowledge in the field.

The relative sliding distance of internal and external screws must be adjusted according to different bone sizes. The bone can be the femoral neck, calcaneus, finger bone, proximal humerus, scaphoid or talus. In some embodiments, the maximum sliding distance of the internal screw compared to the external screw is 2 cm. In some embodiments, the adjustable relative sliding distance between the internal and external screws is 0-0.1, 0-0.2, 0-0.3, 0-0.4, 0-0.5, 0-0.6, 0-0.7, 0-0.8, 0-0.9, 0-1, 0-1.1, 0-1.2, 0-1.3, 0-1.4, 0-1.5, 0-1.6, 0-1.7, 0-1.8, 0-1.9, 0-2.0 cm, and the minimum unit for adjusting the relative distance between the internal screw and the external screw each time is 0.1 mm. In some embodiments, the sliding distance of the internal screw compared to the external screw may be adjusted to 0-1.51, 0-1.73 cm. In some embodiments, the maximum sliding distance between the internal screw and the external screw may be 7, 8, 9, 10, 11, 12 or 13 cm. In some embodiments, the adjustable relative sliding distance between the internal and external screws is 0-7, 0-8, 0-9, 0-10, 0-11, 0-12 or 0-13 cm, and the smallest unit for adjusting the relative distance between internal and external screws is 0.1 mm.

In some implementations, the internal screw comprises a second continuous protrusion part and a first pushing rod section, where the first pushing rod section includes a first thread section, and the first thread section includes external screw threads. Meanwhile, the length adjuster may be designed to include a knob and a channel, where the channel may further include an inner side, the inner side includes a second thread section, and the second thread section includes internal screw threads. The external screw threads of the internal screw have the same screw thread diameter and pitch as the internal screw threads of the length adjuster, so the external screw thread may be screwed and compatible with the internal thread of the length adjuster.

Since the knob of the length adjuster 103 may not enter into the second region 1015, the relative distance between the inner screw 102 and the outer screw 101 may be fixed to prevent the inner screw 102 and the outer screw 101 from sliding again. Both the inner screw 102 and the outer screw 101 with fixed relative distance are assembled outside the body. Finally, the second continuous protrusion part 1021 of the inner screw 102 is rotated clockwise into the portion of the femoral neck toward the femoral head. With reference to FIG. 5 and FIG. 6. Step S4, removing the length adjuster. In one embodiment, after the inner screw 102 and the outer screw 101 are inserted into the femoral neck, the length adjuster 103 may be removed after the relative distance between the inner screw 102 and the outer screw 101 are fixed, to complete the assembly of the sliding device for bone fracture 10. In one embodiment, after the inner screw 102 and the outer screw 101 are inserted into the femoral neck, the length adjuster 103 may not be removed after the relative distance between the inner screw 102 and the outer screw 101 are fixed, to complete the assembly of the sliding device for bone fracture 10. Compared to not using the length adjuster of the present invention, only manual sliding of the relative distance between the inner nail and the outer nail will cause operational limitations. However, with the use of the length adjuster of this invention, the length adjuster can accurately present that the minimum unit for adjusting the relative distance between the inner screw and the outer screw each time is 0.1 mm. Therefore, the relative sliding distance between the inner screw 102 and the outer screw 101 may be adjusted more precisely by using the length adjuster 103. In addition, the groove 1026 of the inner screw is set to accommodate the protrusion 1016 of the first inner side 1013 of the outer screw to ensure that when the femoral neck is shortened, the inner screw 102 near the femoral head slides towards the outer screw 101 which is near the femoral neck and avoids rotational displacement relative to each other.

In one implementation, the sliding device for bone fracture 10 of the present invention adjusts the relative distance between the inner screw 102 and the outer screw 101 to respond to shortening of the femoral neck. When the femoral neck is shortened, the sliding device for bone fracture 10 is compressed due to the shortening that occurs during femoral neck healing, causing the inner screw 102 and the outer screw 101 move towards each other. It prevents the sliding device for bone fracture from protruding through the bone due to impaction generated at the fracture site during the healing process. Additionally, in one implementation, the sliding device for bone fracture 10 can be designed as a buried screw to avoid the phenomenon of traditional compression that the bone screw fixation may cause the bone screw retreat during the healing process of the femoral neck, causing the bone screw to irritate muscle and ligament tissue.

From the present disclosure, it is manifested that various techniques may be used for implementing the concepts described in the present disclosure without departing from the scope of those concepts. Moreover, while the concepts have been described with specific reference to certain implementations, a person of ordinary skill in the art would recognize that changes may be made in form and detail without departing from the scope of those concepts. As such, the described implementations are to be considered in all respects as illustrative and not restrictive. It should also be understood that the present disclosure is not limited to the particular implementations described above. Still, many rearrangements, modifications, and substitutions are possible without departing from the scope of the present disclosure.

What is claimed is:

1. A device comprising:
an external screw comprising:
a first channel extending along a longitudinal axis, and the first channel comprising a first inner side comprising at least one protrusion; and
an internal screw comprising:
a first pushing rod section comprising a first threaded section and at least one groove, wherein the at least one groove is configured to accommodate the at least one protrusion of the first inner side, and the first pushing rod section is configured to slide within the first channel along the longitudinal axis.

2. The device according to claim 1, wherein the first pushing rod section of the internal screw further comprises a second channel, the second channel comprises a second inner side, and the first threaded section is located on the second inner side.

3. The device according to claim 2, wherein the second channel is configured to accommodate a length adjuster, the length adjuster comprises a knob, a smooth section and a second threaded section, and the second threaded section is compatible with the first threaded section.

4. The device according to claim 3, wherein the first channel comprises a first region and a second region, a diameter of the first region is greater than a diameter of the second region, and the diameter of the second region is smaller than a diameter of the knob.

5. The device according to claim 1, wherein the external screw comprises a first continuous protrusion part, and the internal screw comprises a second continuous protrusion part.

6. The device according to claim 5, wherein the first continuous protrusion part and the second continuous protrusion part comprise screw threads.

7. The device according to claim 5, wherein a first spacing of the first continuous protrusion part is different from a second spacing of the second continuous protrusion part.

8. The device according to claim 7, wherein a spacing ratio of the first continuous protrusion part to the second continuous protrusion part is 1:2.

9. The device according to claim 1, wherein the first inner side in the first channel forms a circumference, and a plurality of protrusions is evenly distributed on the circumference.

10. The device according to claim 9, wherein the plurality of protrusions comprises four protrusions.

11. The device according to claim 1, wherein the first pushing rod section of the internal screw forms a circumference, and a plurality of grooves is distributed evenly on the circumference comprises.

12. The device according to claim 11, wherein the plurality of grooves comprises four grooves.

13. The device according to claim 1, wherein an adjustable sliding distance of the internal screw relative to the external screw is no more than 2 cm.

14. The device according to claim 1, wherein the first thread section of the first pushing rod section comprises external screw threads.

15. The device according to claim 1, wherein a material of the internal screw and the external screw comprises Ti-6Al-4V.

16. The device according to claim 1, wherein a material of the internal screw and the external screw comprises stainless steel.

17. An assembling method, comprising:
providing a device, the device comprising an internal screw and an external screw, the external screw comprising a first channel configured to accommodate a length adjuster, a first inner side of the first channel comprising at least one protrusion, the internal screw comprising a first pushing rod section with at least one groove, the first pushing rod section comprising a second channel, a second inner side of the second channel comprising internal screw threads, the at least one groove configured to accommodate the at least one protrusion;
inserting the first pushing rod section of the internal screw into the first channel of the external screw, the internal screw and the external screw being slidable relative to each other;
placing the length adjuster in the first channel of the external screw, the length adjuster being provided with a second thread section which comprises external screw threads, and the external screw threads on the second thread section being made compatible with the internal screw threads of the first pushing rod section;
rotating the length adjuster to adjust a sliding distance of the internal screw relative to the external screw; and
removing the length adjuster.
